(19) United States
(12) Patent Application Publication
(10) Pub. No.: US 2026/0306079 A1
(43) Pub. Date: Oct. 8, 2026

(54) SELF-CONTAINED SHAVE BIOPSY COLLECTION AND STORAGE DEVICE

(71) Applicant: ClickPath Biopsy Solutions, Inc., Los Angeles, CA (US)

(72) Inventors: Graham Chester Arthur Ives, Los Angeles, CA (US); Alan Lee Viosca, Seattle, WA (US); Jedediah J. McCann, Los Angeles, CA (US)

(73) Assignee: ClickPath Biopsy Solutions, Inc., Los Angeles, CA (US)

(21) Appl. No.: 19/462,273

(22) Filed: Jan. 28, 2026

Related U.S. Application Data
(60) Provisional application No. 63/751,911, filed on Jan. 31, 2025.

(51) Int. Cl. A61B 10/00; A61B 10/02

(52) U.S. Cl. CPC A61B 10/0096; A61B 10/0266; A61B 2010/0208

(57) ABSTRACT

A device for collecting a biopsy sample and methods of use. The device may include: a first end comprising a tissue chamber in fluid communication with an opening configured to receive a tissue, such that the tissue extends at least partially into the tissue chamber; a fluid chamber containing a storage medium, the fluid chamber in fluid communication with the tissue chamber; a blade adjacent the opening; and an actuator. When triggered, the actuator may be configured to cause: the blade to move from a first position in which the opening is unobstructed, across the opening, and to a second position in which the opening is obstructed by a portion of the blade, thereby cutting a sample from the tissue and sealing the sample within the tissue chamber; and the fluid chamber to release the storage medium into the tissue chamber when the blade is in the second position.

### CROSS-REFERENCE TO RELATED APPLICATIONS

This application claims benefit to U.S. Prov. Pat. App. No. 63/751,911 filed on Jan. 31, 2025. The entirety of this application is incorporated herein by reference.

### TECHNICAL FIELD

The present disclosure relates generally to methods, systems, and apparatuses related to collecting a biopsy sample. More particularly, the present disclosure relates to a device capable of collecting a shave biopsy sample from a tissue and storing the biopsy sample in a storage medium within a sealed chamber of the device, thereby enabling safe transport to a testing facility. The disclosed techniques may be applied to, for example, biopsy of dermal tissues and other external tissues.

### BACKGROUND

Performing biopsies, including but not limited to dermal tissue biopsies, may be a beneficial tool for detecting health conditions and issues as well as improving overall wellness awareness. For example, shave biopsies may be performed on skin tissue to screen for cancer and other conditions that may require medical attention. Biopsies can also be a useful tool in developing personalized care plans, e.g., for medical and/or cosmetic care.

Conventional shave biopsy techniques involve the use of a scalpel or flexible razor blade to manually shave the skin of the patient over the desired area and to the desired depth to release a sample from the tissue. Typically, a physician can directly perform the biopsy on a patient, or the patient may need to visit a care center for the biopsy to be performed by a trained technician. After collection, the sample may be preserved in a storage medium (e.g., a fixative) within a sealed container such that it may be transported to a testing facility for assessment. However, conventional techniques may create difficulties for patients because of the need to visit a physician or care center in person for collection of the biopsy. Additionally, even when performed by a physician or technician, the conventional techniques require sufficient training and experience to be performed correctly. Incorrect technique, e.g., inaccurate sample area or depth, can lead to errors in the assessment, including misdiagnoses. Furthermore, adequate storage is required to enable proper assessment of the sample at a testing facility and avoid the need to repeat the biopsy.

As such, it is important to collect biopsy samples in a convenient, safe, and accurate manner as part of a health assessment. Accordingly, it would be advantageous to have a device to enable convenient, consistent, and accurate biopsy sample collection and storage thereof.

### SUMMARY

Described herein are devices for collecting a biopsy sample and methods of use. The devices may include: a first end comprising a tissue chamber in fluid communication with an opening configured to receive a tissue, such that the tissue extends at least partially into the tissue chamber; a fluid chamber containing a storage medium, the fluid chamber in fluid communication with the tissue chamber; a blade adjacent the opening; and an actuator. When triggered, the actuator may be configured to cause: the blade to move from a first position in which the opening is unobstructed, across the opening, and to a second position in which the opening is obstructed by a portion of the blade, thereby cutting a sample from the tissue and sealing the sample within the tissue chamber; and the fluid chamber to release the storage medium into the tissue chamber when the blade is in the second position.

### BRIEF DESCRIPTION OF THE DRAWINGS

The accompanying drawings, which are incorporated in and form a part of the specification, illustrate the examples of the invention and together with the written description serve to explain the principles, characteristics, and features of the invention. Various aspects of at least one example are discussed below with reference to the accompanying drawings, which are not intended to be drawn to scale. In the drawings:

FIG. 1 depicts a diagram of an exemplary device for collecting a shave biopsy sample, according to an example of the present disclosure;

FIG. 2A depicts another exemplary device for collecting a shave biopsy sample, according to an example of the present disclosure;

FIG. 2B depicts aspects of the exemplary device shown in FIG. 2A, according to an example of the present disclosure;

FIG. 3A depicts another exemplary device for collecting a shave biopsy sample, according to an example of the present disclosure;

FIGS. 3B-3D depict aspects of the exemplary device shown in FIG. 3A, according to an example of the present disclosure;

FIG. 4A depicts a perspective view of another exemplary device for collecting a shave biopsy sample in an unactuated state, according to an example of the present disclosure;

FIG. 4B depicts a perspective view of the exemplary device shown in FIG. 4A in an actuated state, according to an example of the present disclosure;

FIG. 4C depicts a cross-section view of the exemplary device shown in FIG. 4A in an unactuated state, according to an example of the present disclosure;

FIG. 4D depicts a cross-section view of the exemplary device shown in FIG. 4A in a first partially actuated state, according to an example of the present disclosure;

FIG. 4E depicts a cross-section view of the exemplary device shown in FIG. 4A in a second partially actuated state, according to an example of the present disclosure;

FIG. 4F depicts a cross-section view of the exemplary device shown in FIG. 4A in the actuated state, according to an example of the present disclosure;

FIG. 4G depicts a planar view of the exemplary device shown in FIG. 4A with a cap in a closed state, according to an example of the present disclosure;

FIG. 4H depicts a planar view of the exemplary device shown in FIG. 4A with a cap in an open state, according to an example of the present disclosure; and

FIG. 5 depicts a flow chart of an illustrative method of collecting a shave biopsy sample using a device as disclosed herein, according to an example of the present disclosure.

### DETAILED DESCRIPTION

This disclosure is not limited to the particular systems, devices and methods described, as these may vary. The terminology used in the description is for the purpose of describing the particular versions or examples only, and is not intended to limit the scope. Such aspects of the disclosure be embodied in many different forms; rather, these examples are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample examples, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios of a composition are based upon the total weight of the composition and all measurements made are at about 25 ºC, unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the example of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and refer to any living organism which contains neural tissue. As such, the terms “patient” and “subject” may include, but are not limited to, any non-human mammal, primate or human. A subject can be a mammal such as a primate, for example, a human. The term “subject” includes domesticated animals (e.g., cats, dogs, etc.); livestock (e.g., cattle, horses, swine, sheep, goats, etc.), and laboratory animals (e.g., mice, rabbits, rats, gerbils, guinea pigs, possums, etc.). A patient or subject may be an adult, child or infant.

The term “tissue” refers to any aggregation of similarly specialized cells which are united in the performance of a particular function.

The term “disorder” is used in this disclosure to mean, and is used interchangeably with, the terms “disease,” “condition,” or “illness,” unless otherwise indicated.

Unless defined otherwise, all technical and scientific terms used herein have the same meanings as commonly understood by one of ordinary skill in the art. Nothing in this disclosure is to be construed as an admission that the examples described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

As discussed herein, it would be advantageous to have a device capable of convenient, consistent, and accurate shave biopsy sample collection as well as safe storage of the collected sample. Ideally, the device is self-contained such that the biopsy sample may be sealed and stored in a storage medium within the device after collection therewith. In some examples, the device may be utilized by technicians and/or physicians. Additionally, in some examples, the device may be utilized by patients such that the biopsy may be performed at any suitable location and safely transported to a testing facility within the device.

The devices and methods disclosed herein aim to simplify the process, improve the reliability, and improve the comfort associated with obtaining a shave biopsy. The devices disclosed herein may not require power to operate and may be self-contained. Furthermore, the devices may be formed as disposable for one-time use. Accordingly, the devices disclosed herein utilize a manual mechanism to create suction to draw an area of human skin of a defined area and depth into an aperture, which may then be transected by a spring-loaded cutting blade. The tissue sample may be immersed within a storage medium within the same device for transport to a pathology lab or tissue culture facility.

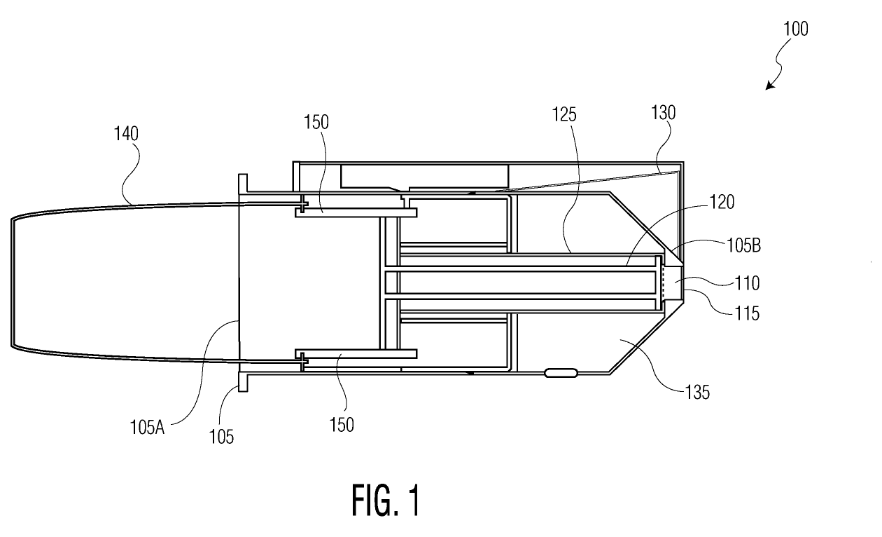

Referring now to FIG. 1, a cross-section view of a shave biopsy device 100 for collecting a shave biopsy sample according to a first example are shown. The device 100 may include a housing 105, a plunger 120, a blocking plate 125, a blade 130, a fluid chamber 135, and an actuator 140. The plunger 120, the blocking plate 125, and/or the fluid chamber 135 may be disposed within the housing 105. The actuator 140 may be configured to operate the plunger 120, the blade 130, and/or the fluid chamber 135 to collect a biopsy sample and store the biopsy sample within the housing 105.

The housing 105 may include a first end 105A and a second end 105B. The housing 105 may define a tissue chamber 110 extending at least partially between the first end 105A and the second end 105B. The tissue chamber 110 may extend from the second end 105B towards the first end 105A. The tissue chamber 110 may be formed as a channel or cavity within the housing 105. The tissue chamber 110 may be shaped according to the housing 105. For example, where the housing 105 is substantially cylindrical, the tissue chamber 110 may be substantially cylindrical. However, the tissue chamber 110 may be formed in a variety of three-dimensional shapes within the housing 105. In an example, the tissue chamber 110 may extend partially between the first end 105A and the second end 105B. In another example, the tissue chamber 110 may extend the entire distance or substantially the entire distance between the first end 105A and the second end 105B. However, it should be understood that one or more portions of the tissue chamber 110 may be filled or blocked by components of the device 100 as further described herein.

The housing 105 may include an opening 115. The opening 115 may be configured to receive a tissue for which a biopsy sample is to be collected. For example, in order to obtain a dermal biopsy sample from an area of interest, the area of interest may be placed over the opening 115. The opening 115 may be disposed at the second end 105B of the housing 105.

The opening 115 may be in fluid communication with the tissue chamber 110. For example, the opening 115 may extend through the second end 105B of the housing 105 and to the tissue chamber 110 in order to couple the tissue chamber 110 to an external environment such that the tissue chamber 110 may receive a tissue. In an example, the opening 115 may have a diameter of approximately 2 mm to approximately 10 mm. However, the opening 115 may be formed in a variety of sizes (e.g., from less than approximately 1 mm to greater than approximately 20 mm). The opening 115 may be sized to obtain a particular clinically useful size of biopsy sample. For example, the size of the biopsy sample may be determined based on the type of tissue and/or the type of conditions and/or disorders that will be assessed using the obtained biopsy sample. Accordingly, the device 100 may be manufactured in multiple types having different sizes of, at least, the opening 115. In some examples, the size of the opening 115 may be adjustable. For example, the size of the opening 115 may be adjusted to provide the correct size for a particular clinical application. In another example, the size of the opening 115 may be adjusted based on the size of a particular area of interest (e.g., a lesion) in order to capture a sufficient portion of the area of interest (e.g., a center of the lesion).

In an example, the opening 115 may include a rim (not shown) configured to facilitate an air-tight seal with the tissue pressed against the opening 115. In another example, the opening 115 may include a feature (not shown), such as an adhesive, a soft gasket, or another feature for facilitating an air-tight seal.

The plunger 120 may be at least partially disposed within the tissue chamber 110. The plunger 120 may be in fluid communication with the opening 115. The plunger 120 may be configured to provide suction at the opening 115. For example, the plunger 120 may be withdrawn towards the first end 105A and away from the second end 105B, thereby creating suction at the opening 115. In other examples, another movement of the plunger 120 may generate suction at the opening 115. As such, when a tissue is positioned over the opening 115, the plunger 120 may be operated to create suction to draw the tissue through the opening 115 and partially or fully within the tissue chamber 110.

The blocking plate 125 may be disposed within the tissue chamber 110. The blocking plate 125 may be arranged between the second end 105B and the plunger 120. The blocking plate 125 may be perforated, gas-permeable, or otherwise configured to enable fluid communication through the blocking plate 125 such that the plunger 120 is in fluid communication with the opening 115 as described and shown.

The blocking plate 125 may be configured to contact the tissue when the tissue is drawn into the tissue chamber 110. For example, the blocking plate 125 may be disposed at a predetermined distance from the opening 115 such that a tissue reaches and contacts the blocking plate 125 when drawn into the tissue chamber 110. In an example, the blocking plate 125 may be configured to compress the tissue when drawn into the tissue chamber 110. For example, when the tissue is pressed against the opening 115 and drawn into the tissue chamber 110 by the suction from the plunger 120, the tissue may contact the blocking plate 125 and continue to be drawn further into the tissue chamber 110 by the suction. The tissue may compress against the blocking plate 125. The compression against the blocking plate 125 may be clinically advantageous by enabling the collection of a thin tissue sample. For example, a substantially thin sample may be desirable to collect an adequate sample for assessment of the area of interest while minimizing damage to the tissue and/or bleeding.

The blocking plate 125 may be arranged at a fixed depth, i.e., a fixed distance from the opening 115. The fixed depth may be selected to enable the tissue to contact the blocking plate 125 and/or compress against the blocking plate 125 when drawn into the tissue chamber 110. Furthermore, the fixed depth may be selected to obtain a sufficient depth of the sample for clinical purposes. For example, a biopsy depth of at least about 0.74 mm may be useful for some diagnostic purposes (e.g., diagnosis of melanoma). Assessment of a shallower biopsy sample may lead to inconclusive results and/or misdiagnosis. Accordingly, the fixed depth may be approximately 0.74 mm, approximately 1 mm, or greater. In other examples, the fixed depth may range from less than approximately 0.5 mm to greater than approximately 3 mm. Additional depths may be selected as would be known to a person having an ordinary level of skill in the art. For example, the depth may be selected based on the type of tissue and/or the type of conditions and/or disorders that will be assessed using the obtained biopsy sample.

In an example, the blocking plate 125 may have an adjustable depth. For example, a user-operated mechanism may be provided on the device 100 to allow movement and/or locking of the depth of the blocking plate 125. Accordingly, the depth may be selected and the blocking plate 125 may be adjusted by a user based on the type of tissue and/or the type of conditions and/or disorders that will be assessed using the obtained biopsy sample. For example, clinical guidelines may be referenced in order to determine a suitable depth for the biopsy sample based on the particular clinical application.

The blocking plate 125 may be configured to be moved, withdrawn, or retracted after collection of the biopsy sample. For example, while the blocking plate 125 is useful for accurate and consistent collection of the biopsy sample, it may be further advantageous to withdraw the blocking plate 125 after cutting the sample from the tissue in order to allow the sample to advance into the tissue chamber 110 (e.g., further towards the first end 105A) for storage. Accordingly, the blocking plate 125 may be configured to retract away from the second end 105B after collection of the sample. For example, the blocking plate 125 may withdraw towards the first end 105A.

In another example, the blocking plate 125 may rotate and/or translate to a lateral side (i.e., orthogonal to the axis between the first end 105A and the second end 105B) in order to open additional space within the tissue chamber 110 for storage of the sample. The blocking plate 125 may perform a combination of the described movements in order to open additional space within the tissue chamber 110 for storage of the sample.

The blocking plate 125 may include one or more components configured to sense contact and/or pressure from the tissue against the blocking plate 125 in order to trigger an action by the device 100. For example, the blocking plate 125 may include a mechanism for sensing contact and/or pressure such that, upon a threshold amount of contact and/or pressure, the blade 130 is operated to cut the tissue as further described herein. Accordingly, the mechanism may ensure that the tissue is sufficiently received within the opening 115 prior to operating the blade 130. The mechanism for sensing the contact and/or pressure may comprise a spring mechanism, a loaded trigger, a sensor, or any other mechanism known and understood by a person having an ordinary level of skill in the art for the purpose as described.

The blocking plate 125 may be formed with a cylindrical shaft in the same manner as the plunger 120. The cylindrical shaft of the blocking plate 125 may be arranged in a telescoping manner with the plunger 120. For example, the cylindrical shaft of the blocking plate 125 may form an outer telescoping element and the plunger 120 may form an inner telescoping element such that each element can be independently advanced or withdrawn without interfering with one another.

The cylindrical shaft of the blocking plate 125 may have retaining pins (not shown) for locking the position of the blocking plate 125 before and during collection of the tissue sample. After collection of the tissue sample, the retaining pins 150 may be released as further described herein to enable the blocking plate 125 to be retracted.

The blade 130 may be disposed at the second end 105B of the housing 105. The blade 130 may be disposed adjacent to the opening 115. For example, the blade 130 may be disposed outside of the tissue chamber 110 in a location adjacent to the opening 115.

The blade 130 may be configured to move from a first position to a second position. The blade 130 may move from a first position across the opening 115 to a second position. In an example, when a tissue is positioned over the opening 115 and at least partially drawn within the tissue chamber 110, the movement of the blade 130 may cut the portion of the tissue drawn within the tissue chamber 110. A sample may be released from the tissue into the tissue chamber 110.

In an example, the blade 130 may seal the opening 115 when placed in the second position. For example, after cutting the sample from the tissue and reaching the second position, the blade 130 may block the opening 115. The blade 130 may form a sufficient seal over the opening 115 (e.g., a hermetic seal). The housing 105 may include a slot or groove configured to receive the blade 130. The blade 130 may be received within the slot or groove and may be held in a stable manner in the second position to seal the opening 115. Accordingly, the blade 130 may seal the tissue chamber 110 after cutting the sample from the tissue, thereby enclosing the sample within the sealed tissue chamber 110.

In another example, the opening 115 may be sealed by additional mechanisms. For example, a sealing cap may be placed over the opening 115 to securely seal the opening 115. A combination of the blade 130 and a sealing cap may be used to seal the opening 115 after collection of the tissue sample.

The fluid chamber 135 may be in fluid communication with the tissue chamber 110. The fluid chamber 135 may be connected to the tissue chamber 110 by a fluid channel that may be opened or closed to control the flow of fluid from within the fluid chamber. In an example, the fluid chamber 135 may be contained within the housing 105. In another example, the fluid chamber 135 is outside of the housing.

The fluid chamber 135 may contain a storage medium. For example, the storage medium may be one or more of a fixative, a preservative, a cell culture medium, a growth medium, formalin, and additional types of mediums that may be useful for storing a biopsy sample. The storage medium may be maintained within the fluid chamber 135 and partitioned from the remainder of the tissue chamber 110.

After collection of the tissue sample and sealing of the opening 115, the storage medium may be released from the fluid chamber 135 into the remainder of the tissue chamber 110 to expose the tissue sample therein to the storage medium. In an example, movement of an actuator 140 and/or the plunger 120 may automatically release the storage medium from the fluid chamber 135. In an example, the actuator 140 may be a button or a trigger. The actuator 140 may be disposed on a side of the housing 105 between the first end 105A and the second end 105B. However, it should be understood that various types of actuators 140 and placements or arrangements of the actuator 140 are possible within the scope of this disclosure. For example, a threshold amount of movement may cause the fluid chamber 135 to be opened to release the storage medium from the fluid chamber 135 and/or for a sufficient pressure to draw out the storage medium from the fluid chamber 135. Accordingly, the storage medium will flow into the tissue chamber 110.

Additionally or alternatively, retraction of the blocking plate 125 may automatically release the storage medium from the fluid chamber 135. For example, the blocking plate 125 may initially block a port communicating between the fluid chamber 135 and the tissue chamber 110. As the blocking plate 125 is retracted, the port may be uncovered such that the storage medium flows out of the fluid chamber 135 and into the tissue chamber 110.

The storage medium may flow into the tissue chamber 110 based on the pressure in the fluid chamber 135 compared to the pressure in the tissue chamber. For example, the fluid chamber 135 may be pressurized. In an example, the fluid chamber 135 may include an additional mechanism to release the storage medium , e.g., a pumping mechanism (not shown). In another example, the device 100 may be shaken to release the storage medium. Accordingly, the tissue sample is contacted and stored in the storage medium within the tissue chamber 110.

Upon triggering the actuator 140, the actuator 140 may operate one or more components of the device 100. In some examples, triggering the actuator 140 may cause the plunger to withdraw towards the first end 105A of the housing 105, thereby creating suction and drawing a tissue at the opening 115 at least partially within the tissue chamber 110. Additionally or alternatively, the actuator 140 may cause the blade 130 to move from the first position to the second position to cut the sample from the tissue. The actuator 140 may cause the blocking plate 125 to withdraw away from the second end 105B of the housing 105. The actuator 140 may cause the fluid chamber 135 to release the storage medium into the tissue chamber 110 to expose the sample to the storage medium. The actuator 140 may cause the device 100 to perform a plurality of the described functions or all of the described functions.

Triggering the actuator 140 may cause one or more of the described steps to occur sequentially. For example, the blade 130 may be operated prior to the release of the storage medium from the fluid chamber 135. In another example, the actuator 140 may cause one or more of the described steps to occur simultaneously. For example, the blade 130 may be operated while the plunger 120 is being withdrawn to draw the tissue within the tissue chamber 110. Likewise, the blocking plate 125 may be withdrawn while the storage medium is released from the fluid chamber. However, it should be understood that any permutation of the described steps performed either sequentially and/or simultaneously that is sufficient to collect a tissue sample as described herein in the view of a person having an ordinary level of skill in the art is contemplated within the scope of this disclosure.

In another example, triggering the actuator 140 may cause each of the described steps to occur. For example, the actuator 140 may trigger the plunger 120 to move or withdraw, and the remaining steps (i.e., operation of the blade 130, withdrawal of the blocking plate 125, and release of the storage medium from the fluid chamber 135) may occur as a chain reaction because the mechanisms may be linked to one another. The remaining steps may be individually triggered by separate mechanisms (e.g., the operation of the blade 130 may be triggered by sufficient contact or pressure against the blocking plate 125 as described herein).

In another example, several or all steps may be individually triggered by the actuator 140. Each step may be triggered by a single movement of the actuator 140. A first set of steps may be triggered by a first actuation of the actuator 140 and a second set of steps may be triggered by a second actuation of the actuator 140. For example, the first actuation may be a first movement such as an initial partial depression of the actuator 140 and the second actuation may be a second movement such as an additional partial depression (or complete depression) of the actuator 140. In another example, the first actuation may be a first movement such as an initial partial or full depression of the actuator 140 and the second actuation may be a second movement such as release of the actuator 140. In a specific example, the first actuation of the actuator 140 may cause the plunger 120 to move or withdraw, thereby creating suction. Thereafter, the second actuation of the actuator 140 may cause the blade 130 to move to cut the tissue, the blocking plate 125 to withdraw (e.g., by release of the retaining pins 150), and the storage medium to be released from the fluid chamber 135.

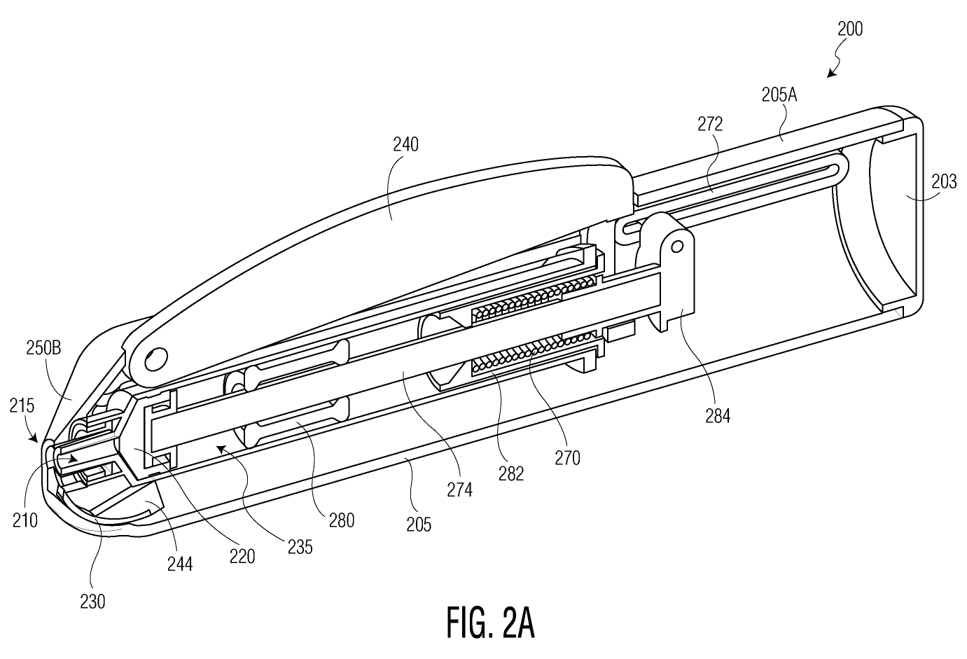

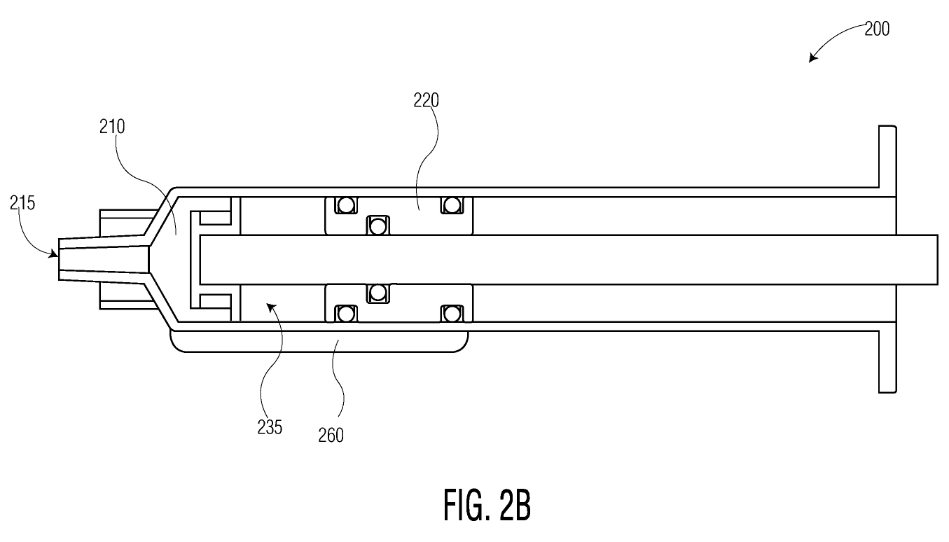

Referring now to FIG. 2A, a cross-sectional view of a shave biopsy device 200 for collecting a shave biopsy sample according to another example is shown. The device 200 may include a housing 205, a top 203, a plunger 220, a plunger shaft 274, a linkage 272, a blade 230, a cutter body 244, a fluid chamber 235, an actuator 240, a stopper 280, a spring 270, a spring housing 282, and a coupler 284. The plunger 220 and/or the fluid chamber 235 may be disposed within the housing 205. The actuator 240 may be configured to operate the plunger 220, the blade 230, and/or the fluid chamber 235 to collect a biopsy sample and store the biopsy sample within the housing 205.

The housing 205 may include a first end 205A and a second end 205B. The housing 205 may define a tissue chamber 210 extending at least partially between the first end 205A and the second end 205B. The tissue chamber 210 may extend from the second end 205B towards the first end 205A. The tissue chamber 210 may be formed as a channel or cavity within the housing 205. The tissue chamber 210 may be shaped according to the housing 205. For example, where the housing 205 is substantially cylindrical, the tissue chamber 210 may be substantially cylindrical. However, the tissue chamber 210 may be formed in a variety of three-dimensional shapes within the housing 205. In an example, the tissue chamber 210 may extend partially between the first end 205A and the second end 205B. In another example, the tissue chamber 210 may extend the entire distance or substantially the entire distance between the first end 205A and the second end 205B. However, it should be understood that one or more portions of the tissue chamber 210 may be filled or blocked by components of the device 200 as further described herein.

The housing 205 may include an opening 215. The opening 215 may be configured to receive a tissue for which a biopsy sample is to be collected. For example, in order to obtain a dermal biopsy sample from an area of interest, the area of interest may be placed over the opening 215. The opening 215 may be disposed at the second end 205B of the housing 205.

The opening 215 may be in fluid communication with the tissue chamber 210. For example, the opening 215 may extend through the second end 205B of the housing 205 and to the tissue chamber 210 in order to couple the tissue chamber 210 to an external environment such that the tissue chamber 210 may receive a tissue. In an example, the opening 215 may have a diameter of approximately 2 mm to approximately 10 mm. However, the opening 215 may be formed in a variety of sizes (e.g., from less than approximately 1 mm to greater than approximately 20 mm). The opening 215 may be sized to obtain a particular clinically useful size of biopsy sample. For example, the size of the biopsy sample may be determined based on the type of tissue and/or the type of conditions and/or disorders that will be assessed using the obtained biopsy sample. Accordingly, the device 200 may be manufactured in multiple types having different sizes of, at least, the opening 215. In some examples, the size of the opening 215 may be adjustable. For example, the size of the opening 215 may be adjusted to provide the correct size for a particular clinical application. In another example, the size of the opening 215 may be adjusted based on the size of a particular area of interest (e.g., a lesion) in order to capture a sufficient portion of the area of interest (e.g., a center of the lesion).

In an example, the opening 215 may include a rim (not shown) configured to facilitate an air-tight seal with the tissue pressed against the opening 215. In another example, the opening 215 may include a feature (not shown), such as an adhesive, a soft gasket, or another feature for facilitating an air-tight seal.

The plunger 220 may be at least partially disposed within the tissue chamber 210. The plunger 220 may be in fluid communication with the opening 215. The plunger 220 may be configured to provide suction at the opening 215. For example, the plunger 220 may be withdrawn towards the first end 205A and away from the second end 205B, thereby creating suction at the opening 215. In other examples, another movement of the plunger 220 may generate suction at the opening 215. As such, when a tissue is positioned over the opening 215, the plunger 220 may be operated to create suction to draw the tissue through the opening 215 and partially or fully within the tissue chamber 210.

The blade 230 may be disposed at the second end 205B of the housing 205. The blade 230 may be disposed adjacent to the opening 215. For example, the blade 230 may be disposed outside of the tissue chamber 210 in a location adjacent to the opening 215.

The blade 230 may be configured to move from a first position to a second position, for example, via movement of the cutter body 244. The cutter body 244 may be coupled to the linkage 272, which may cause the cutter body 244 to move when the actuator 240 is triggered. The blade 230 may move from a first position across the opening 215 to a second position. In an example, when a tissue is positioned over the opening 215 and at least partially drawn within the tissue chamber 210, the movement of the blade 230 may cut the portion of the tissue drawn within the tissue chamber 210. A sample may be released from the tissue into the tissue chamber 210.

In an example, the blade 230 may seal the opening 215 when placed in the second position. For example, after cutting the sample from the tissue and reaching the second position, the blade 230 may block the opening 215. The blade 230 may form a sufficient seal over the opening 215 (e.g., a hermetic seal). The housing 205 may include a slot or groove configured to receive the blade 230. The blade 230 may be received within the slot or groove and may be held in a stable manner in the second position to seal the opening 215. Accordingly, the blade 230 may seal the tissue chamber 210 after cutting the sample from the tissue, thereby enclosing the sample within the sealed tissue chamber 210.

In another example, the opening 215 may be sealed by additional mechanisms. For example, a sealing cap may be placed over the opening 215 to securely seal the opening 215. A combination of the blade 230 and a sealing cap may be used to seal the opening 215 after collection of the tissue sample.

The fluid chamber 235 may be in fluid communication with the tissue chamber 210. The fluid chamber 235 may be connected to the tissue chamber 210 by a fluid channel that may be opened or closed to control the flow of fluid from within the fluid chamber 235. In an example, the fluid chamber 235 may be contained within the housing 205. In another example, the fluid chamber 235 is outside of the housing.

As shown in FIG. 2B, the device 200 may be designed as a dual-chamber or a multi-chamber device whereby the fluid chamber 235 is a separate chamber from the tissue chamber 210. For example, the fluid chamber 235 may be contained within a portion of the device 200 that is separated from the tissue chamber 210. The plunger 220 may include a wide portion that abuts portions of the housing 205 to separate the fluid chamber 235 from the tissue chamber 210. The fluid chamber 235 may be connected to a channel 260, which may be blocked by the plunger 220 in an initial position. As the plunger 220 is depressed during operation of the device, the channel 260 may be unblocked and may communicate with the fluid chamber 235, thereby releasing the storage medium from the channel 260 into the fluid chamber 235 as the device 200 is operated to collect the sample.

The storage medium may be one or more of a fixative, a preservative, a cell culture medium, a growth medium, formalin, and additional types of mediums that may be useful for storing a biopsy sample.

After collection of the tissue sample and sealing of the opening 215, the storage medium may be released from the channel 260 into the fluid chamber 235. In an example, movement of the actuator 240 and/or the plunger 220 may automatically release the storage medium from the fluid chamber 235. In an example, the actuator 240 may be a squeeze mechanism as would be known to a person having an ordinary level of skill in the art. The actuator 240 may be disposed on a side of the housing 205 between the first end 205A and the second end 205B. However, it should be understood that various types of actuators 240 and placements or arrangements of the actuator 240 are possible within the scope of this disclosure. For example, a threshold amount of movement may cause the channel 260 to be opened to release the storage medium into the fluid chamber 235 and/or for a sufficient pressure to draw out the storage medium from the channel 260.

The storage medium may flow into the fluid chamber 235 based on the pressure in the channel 260 compared to the pressure in the fluid chamber 235. For example, the channel 260 may be pressurized. In an example, the fluid chamber 235 may include an additional mechanism to release the storage medium, e.g., a pumping mechanism (not shown). In another example, the device 200 may be shaken to release the storage medium. Accordingly, the tissue sample is contacted and stored in the storage medium within the tissue chamber 210.

Upon triggering the actuator 240, the actuator 240 may operate one or more components of the device 200. In some examples, triggering the actuator 240 may cause the plunger 220 to withdraw towards the first end 205A of the housing 205, thereby creating suction and drawing a tissue at the opening 215 at least partially within the tissue chamber 210. The plunger 220 may move until it is stopped by the stopper 280. Additionally or alternatively, the actuator 240 may cause the cutter body 244 to move and therefore cause the blade 230 to move from the first position to the second position to cut the sample from the tissue. The actuator 240 may cause the channel 260 to release the storage medium into the fluid chamber 235 to expose the sample to the storage medium. The actuator 240 may cause the coupler 284, which is coupled to the actuator 240 to move within the linkage 272. The spring 270 within the spring housing 282 may cause the coupler 284 to move. The movement of the coupler 284 may cause a plunger shaft 274 to move, thereby causing the plunger 220 to move. The actuator 240 may cause the device 200 to perform a plurality of the described functions or all of the described functions.

Triggering the actuator 240 may cause one or more of the described steps to occur sequentially. For example, the blade 230 may be operated prior to the release of the storage medium from the fluid chamber 235. In another example, the actuator 240 may cause one or more of the described steps to occur simultaneously. For example, the blade 230 may be operated while the plunger 220 is being withdrawn to draw the tissue within the tissue chamber 210. However, it should be understood that any permutation of the described steps performed either sequentially and/or simultaneously that is sufficient to collect a tissue sample as described herein in the view of a person having an ordinary level of skill in the art is contemplated within the scope of this disclosure.

In another example, triggering the actuator 240 may cause each of the described steps to occur. For example, the actuator 240 may trigger the plunger 220 to move or withdraw, and the remaining steps may occur as a chain reaction because the mechanisms may be linked to one another. The remaining steps may be individually triggered by separate mechanisms.

In another example, several or all steps may be individually triggered by the actuator 240. Each step may be triggered by a single movement of the actuator 240. A first set of steps may be triggered by a first actuation of the actuator 240 and a second set of steps may be triggered by a second actuation of the actuator 240. For example, the first actuation may be a first movement such as an initial partial depression of the actuator 240 and the second actuation may be a second movement such as an additional partial depression (or complete depression) of the actuator 240. In another example, the first actuation may be a first movement such as an initial partial or full depression of the actuator 240 and the second actuation may be a second movement such as release of the actuator 240. In a specific example, the first actuation of the actuator 240 may cause the plunger 220 to move or withdraw, thereby creating suction. Thereafter, the second actuation of the actuator 240 may cause the blade 230 to move to cut the tissue, and the storage medium to be released from the fluid chamber 235.

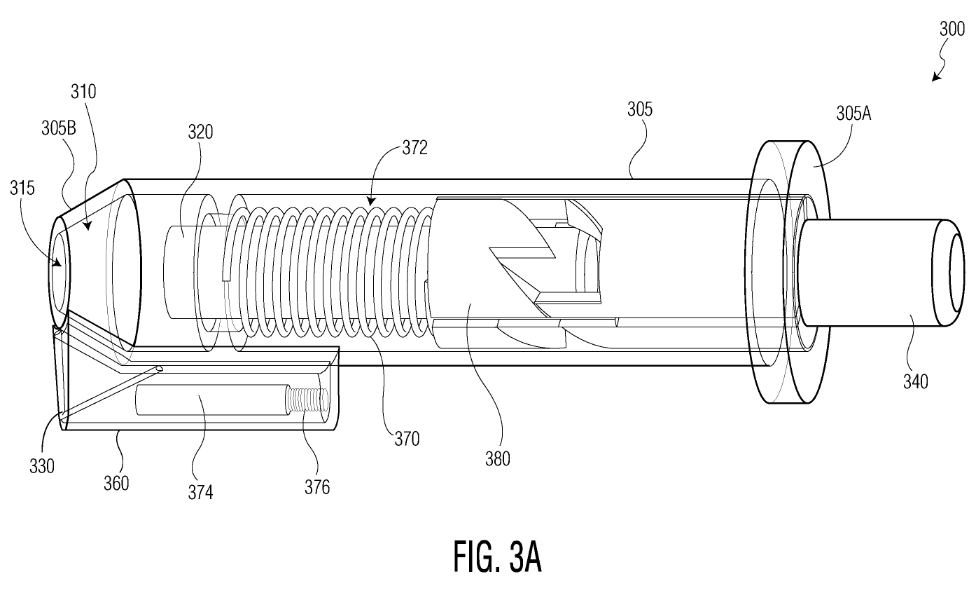

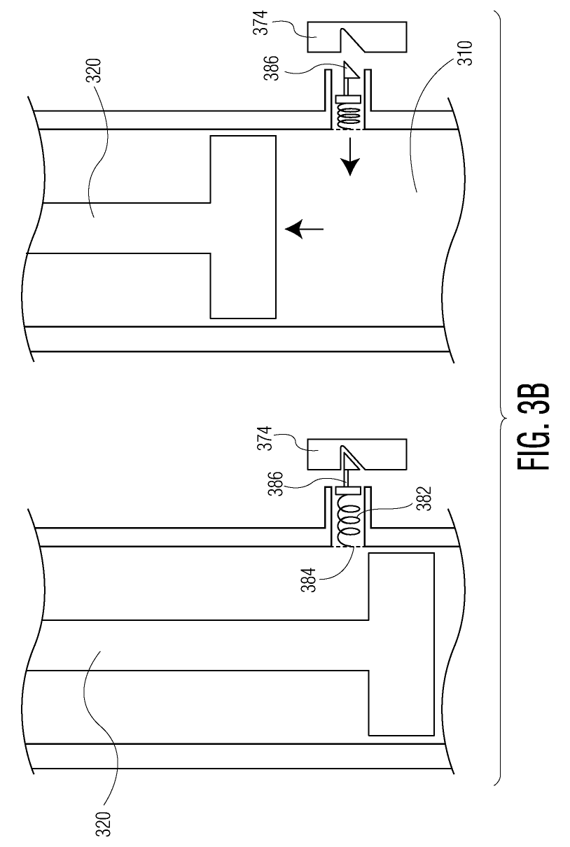

Referring now to FIG. 3A, a perspective view of a shave biopsy device 300 for collecting a shave biopsy sample according to another example is shown. The device 300 may include a housing 305, a plunger 320, a blade 330, an actuator 340, and a rotating cartridge 380. The plunger 320 may be disposed within the housing 305. The actuator 340 may be configured to operate the plunger 320 and/or the blade 330 to collect a biopsy sample and store the biopsy sample within the housing 305.

The housing 305 may include a first end 305A and a second end 305B. The housing 305 may define a tissue chamber 310 extending at least partially between the first end 305A and the second end 305B. The tissue chamber 310 may extend from the second end 305B towards the first end 305A. The tissue chamber 310 may be formed as a channel or cavity within the housing 305. The tissue chamber 310 may be shaped according to the housing 305. For example, where the housing 305 is substantially cylindrical, the tissue chamber 310 may be substantially cylindrical. However, the tissue chamber 310 may be formed in a variety of three-dimensional shapes within the housing 305. In an example, the tissue chamber 310 may extend partially between the first end 305A and the second end 305B. In another example, the tissue chamber 310 may extend the entire distance or substantially the entire distance between the first end 305A and the second end 305B. However, it should be understood that one or more portions of the tissue chamber 310 may be filled or blocked by components of the device 300 as further described herein.

The housing 305 may include an opening 315. The opening 315 may be configured to receive a tissue for which a biopsy sample is to be collected. For example, in order to obtain a dermal biopsy sample from an area of interest, the area of interest may be placed over the opening 315. The opening 315 may be disposed at the second end 305B of the housing 305.

The opening 315 may be in fluid communication with the tissue chamber 310. For example, the opening 315 may extend through the second end 305B of the housing 305 and to the tissue chamber 310 in order to couple the tissue chamber 310 to an external environment such that the tissue chamber 310 may receive a tissue. In an example, the opening 315 may have a diameter of approximately 2 mm to approximately 10 mm. However, the opening 315 may be formed in a variety of sizes (e.g., from less than approximately 1 mm to greater than approximately 20 mm). The opening 315 may be sized to obtain a particular clinically useful size of biopsy sample. For example, the size of the biopsy sample may be determined based on the type of tissue and/or the type of conditions and/or disorders that will be assessed using the obtained biopsy sample. Accordingly, the device 300 may be manufactured in multiple types having different sizes of, at least, the opening 315. In some examples, the size of the opening 315 may be adjustable. For example, the size of the opening 315 may be adjusted to provide the correct size for a particular clinical application. In another example, the size of the opening 315 may be adjusted based on the size of a particular area of interest (e.g., a lesion) in order to capture a sufficient portion of the area of interest (e.g., a center of the lesion).

In an example, the opening 315 may include a rim (not shown) configured to facilitate an air-tight seal with the tissue pressed against the opening 315. In another example, the opening 315 may include a feature (not shown), such as an adhesive, a soft gasket, or another feature for facilitating an air-tight seal.

The plunger 320 may be at least partially disposed within the tissue chamber 310. The plunger 320 may be in fluid communication with the opening 315. The plunger 320 may be configured to provide suction at the opening 315. For example, the plunger 320 may be withdrawn towards the first end 305A and away from the second end 305B, thereby creating suction at the opening 315. In other examples, another movement of the plunger 320 may generate suction at the opening 315. As such, when a tissue is positioned over the opening 315, the plunger 320 may be operated to create suction to draw the tissue through the opening 315 and partially or fully within the tissue chamber 310.

The blade 330 may be disposed at the second end 305B of the housing 305. The blade 330 may be disposed adjacent to the opening 315. For example, the blade 330 may be disposed outside of the tissue chamber 310 in a location adjacent to the opening 315. The blade 130 may be at least partially contained within an additional blade chamber 360 of the housing 305. The blade chamber 360 may house the blade 330 as well as additional components for operating the blade 130 as described herein. For example, the blade chamber 360 may contain a blade actuator 374 and a blade actuator spring 376.

A spring 370 may press the rotating cartridge 380 against the actuator 340. The actuator 340 and the cartridge 380 may have complimentary shapes such that when the actuator 340 is pressed down, the cartridge 380 rotates. When the actuator 340 is released, the rotated cartridge 380 may be pushed up by the spring 370 as it decompresses. Because of the rotation, the cartridge 380 may be able to move up, thereby causing the plunger 320 to create suction within the tissue chamber 310. The space 372 may be fluidly linked to the tissue chamber 310 allowing fluid to contact the tissue sample.

The blade actuator 374 and a blade actuator spring 376 may be triggered by one or more of a button coupled to the actuator 340 and the mechanisms shown in FIGS. 3B-3D. FIG. 3B illustrates a mechanism in which the blade actuator 374 and the blade actuator spring 376 are triggered by a pressure actuated spring block coupled to the tissue chamber 310. A spring 382 may be positioned between a permeable base 384 and a catch 386 that forms an airtight seal. The catch 386 may be configured to fit within an opening of the blade actuator 374. When suction is created in the tissue chamber 310 as the plunger 320 is drawn up, the spring 382 may compress and may pull the catch 386 out of the blade actuator 374, allowing it and the blade 330 to move.

FIG. 3C illustrates a similar concept using a pressure activated diaphragm 390. The diaphragm 390 may form an airtight seal of the tissue chamber 310 and may include a protrusion configure to fit within an opening of the blade actuator 374. When suction is created in the tissue chamber 310 as the plunger 320 is drawn up, the diaphragm 390 may move in and the protrusion may out of the blade actuator 374, allowing it and the blade 330 to move.

FIG. 3C illustrates a similar concept using a pressure activated diaphragm 390. The diaphragm 390 may form an airtight seal of the tissue chamber 310 and may include a protrusion configure to fit within an opening of the blade actuator 374. When suction is created in the tissue chamber 310 as the plunger 320 is drawn up, the diaphragm 390 may move in and the protrusion may out of the blade actuator 374, allowing it and the blade 330 to move.

FIG. 3D illustrates a similar concept using a pressure activated lever 392. A first portion of the lever 390 may extend into the tissue chamber 310 via an airtight seal 394. A second portion of the lever 390 may extend into the blade chamber 360. When suction is created in the tissue chamber 310 as the plunger 320 is drawn up, the first portion of the lever 392 may move up and the second portion of the lever 390 may move down. This movement may trigger the blade actuator 374, allowing it and the blade 330 to move.

The blade 330 may be configured to move from a first position to a second position. The blade 330 may move from a first position across the opening 315 to a second position. In an example, when a tissue is positioned over the opening 315 and at least partially drawn within the tissue chamber 310, the movement of the blade 330 may cut the portion of the tissue drawn within the tissue chamber 310. A sample may be released from the tissue into the tissue chamber 310.

In an example, the blade 330 may seal the opening 315 when placed in the second position. For example, after cutting the sample from the tissue and reaching the second position, the blade 330 may block the opening 315. The blade 330 may form a sufficient seal over the opening 315 (e.g., a hermetic seal). The housing 305 may include a slot or groove configured to receive the blade 330. The blade 330 may be received within the slot or groove and may be held in a stable manner in the second position to seal the opening 315. Accordingly, the blade 330 may seal the tissue chamber 310 after cutting the sample from the tissue, thereby enclosing the sample within the sealed tissue chamber 310.

In another example, the opening 315 may be sealed by additional mechanisms. For example, a sealing cap may be placed over the opening 315 to securely seal the opening 315. A combination of the blade 330 and a sealing cap may be used to seal the opening 315 after collection of the tissue sample.

In an example, one or more of an interior of the plunger 320 and a space 372 surrounding the plunger 320 may serve as a fluid chamber. The fluid chamber may be in fluid communication with the tissue chamber 310. The fluid chamber may be connected to the tissue chamber 310 by a fluid channel that may be opened or closed to control the flow of fluid from within the fluid chamber. In an example, the fluid chamber may be contained within the housing 305. In another example, the fluid chamber may be outside of the housing.

The fluid chamber may contain a storage medium. For example, the storage medium may be one or more of a fixative, a preservative, a cell culture medium, a growth medium, formalin, and additional types of mediums that may be useful for storing a biopsy sample. The storage medium may be maintained within the fluid chamber and partitioned from the remainder of the tissue chamber 310.

After collection of the tissue sample and sealing of the opening 315, the storage medium may be released from the fluid chamber into the remainder of the tissue chamber 310 to expose the tissue sample therein to the storage medium. In an example, movement of an actuator 340 and/or the plunger 320 may automatically release the storage medium from the fluid chamber 335. In an example, actuator 340 may be formed as part of the plunger 320, e.g., as a unitary component forming the top of the plunger 320 in the manner of a standard syringe plunger However, it should be understood that various types of actuators 340 and placements or arrangements of the actuator 340 are possible within the scope of this disclosure. For example, a threshold amount of movement may cause the fluid chamber to be opened to release the storage medium from the fluid chamber and/or for a sufficient pressure to draw out the storage medium from the fluid chamber 335. Accordingly, the storage medium will flow into the tissue chamber 310.

The storage medium may flow into the tissue chamber 310 based on the pressure in the fluid chamber compared to the pressure in the tissue chamber. For example, the fluid chamber may be pressurized. In an example, the fluid chamber may include an additional mechanism to release the storage medium , e.g., a pumping mechanism (not shown). In another example, the device 300 may be shaken to release the storage medium. Accordingly, the tissue sample is contacted and stored in the storage medium within the tissue chamber 310.

Upon triggering the actuator 340, the actuator 340 may operate one or more components of the device 300. In some examples, triggering the actuator 340 may cause the plunger to withdraw towards the first end 305A of the housing 305, thereby creating suction and drawing a tissue at the opening 315 at least partially within the tissue chamber 310. Additionally or alternatively, the actuator 340 may cause the blade 330 to move from the first position to the second position to cut the sample from the tissue. The actuator 340 may cause the fluid chamber to release the storage medium into the tissue chamber 310 to expose the sample to the storage medium. The actuator 340 may cause the device 300 to perform a plurality of the described functions or all of the described functions.

Triggering the actuator 340 may cause one or more of the described steps to occur sequentially. For example, the blade 330 may be operated prior to the release of the storage medium from the fluid chamber 335. In another example, the actuator 340 may cause one or more of the described steps to occur simultaneously. For example, the blade 330 may be operated while the plunger 320 is being withdrawn to draw the tissue within the tissue chamber 310. However, it should be understood that any permutation of the described steps performed either sequentially and/or simultaneously that is sufficient to collect a tissue sample as described herein in the view of a person having an ordinary level of skill in the art is contemplated within the scope of this disclosure.

In another example, triggering the actuator 340 may cause each of the described steps to occur. For example, the actuator 340 may trigger the plunger 320 to move or withdraw, and the remaining steps may occur as a chain reaction because the mechanisms may be linked to one another. The remaining steps may be individually triggered by separate mechanisms.

In another example, several or all steps may be individually triggered by the actuator 340. Each step may be triggered by a single movement of the actuator 340. A first set of steps may be triggered by a first actuation of the actuator 340 and a second set of steps may be triggered by a second actuation of the actuator 340. For example, the first actuation may be a first movement such as an initial partial depression of the actuator 340 and the second actuation may be a second movement such as an additional partial depression (or complete depression) of the actuator 340. In another example, the first actuation may be a first movement such as an initial partial or full depression of the actuator 340 and the second actuation may be a second movement such as release of the actuator 340. In a specific example, the first actuation of the actuator 340 may cause the plunger 320 to move or withdraw, thereby creating suction. Thereafter, the second actuation of the actuator 340 may cause the blade 330 to move to cut the tissue and the storage medium to be released from the fluid chamber 335.

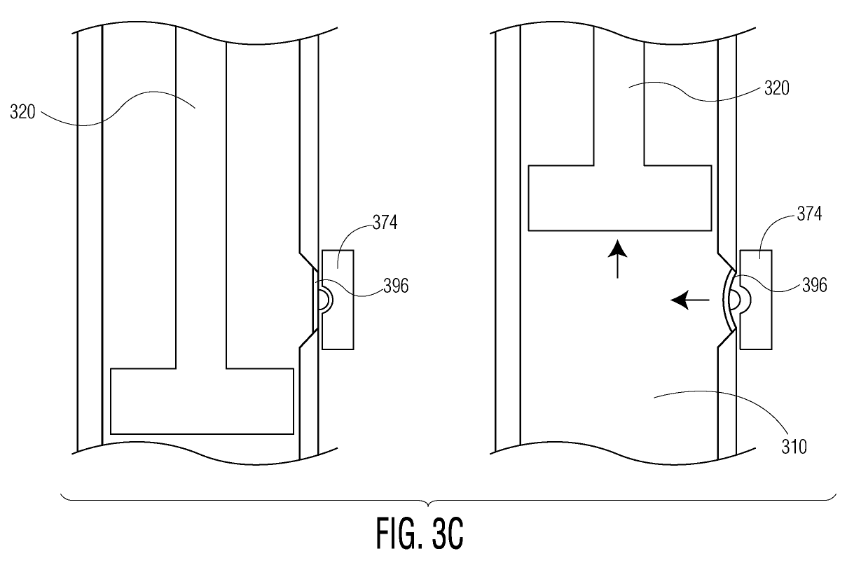

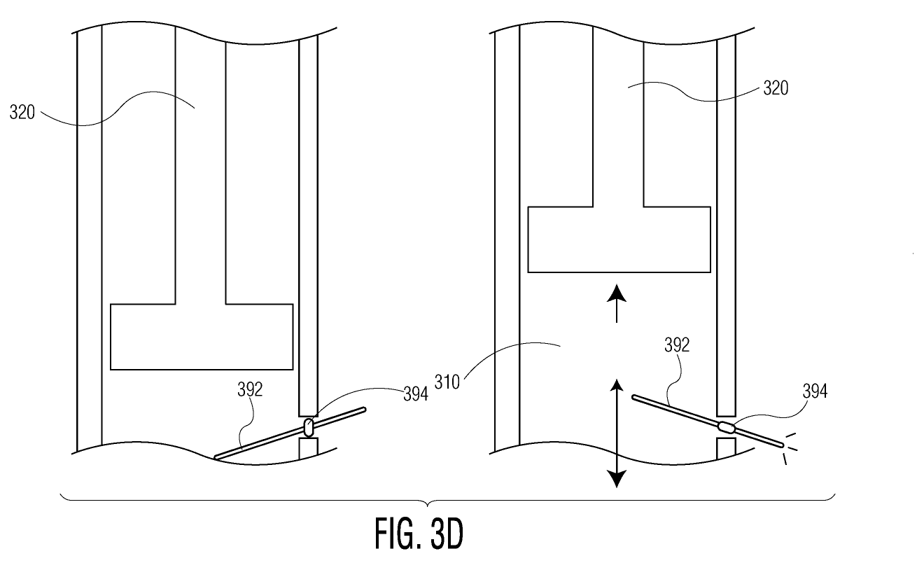

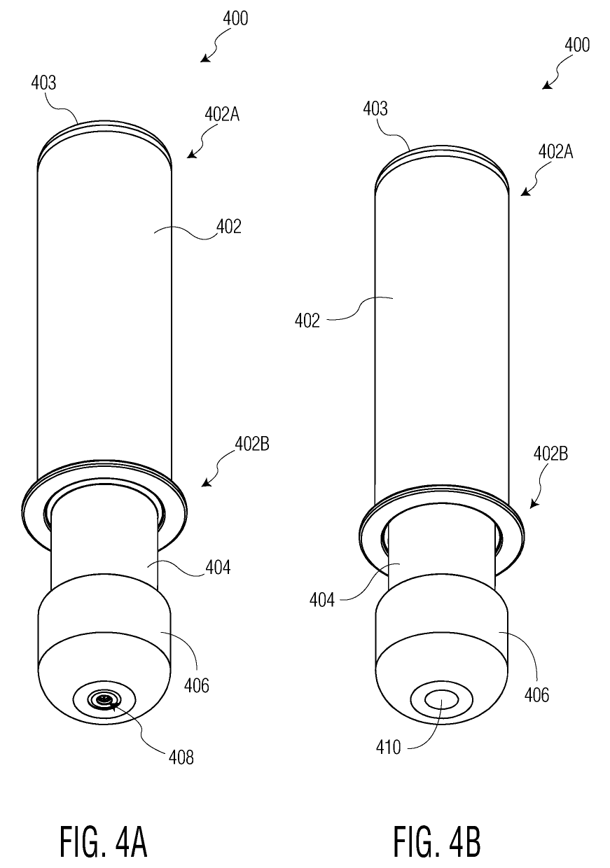

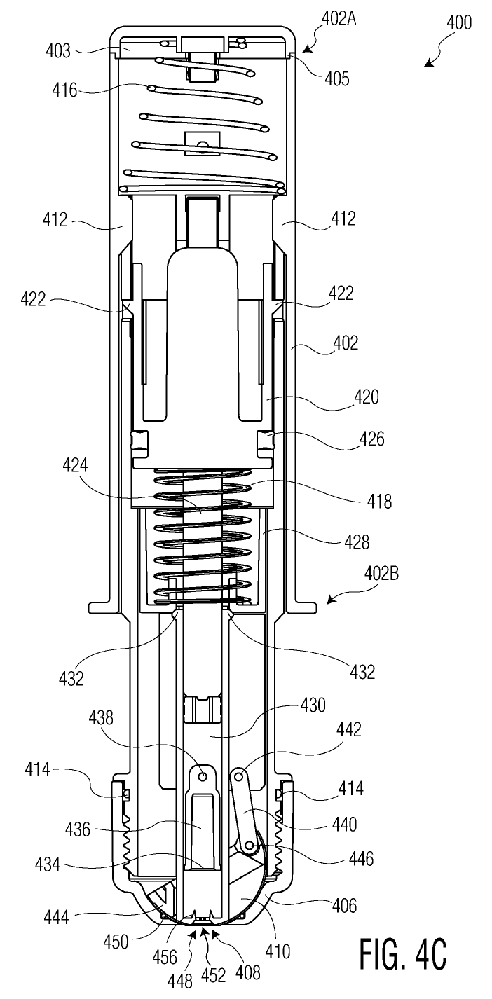

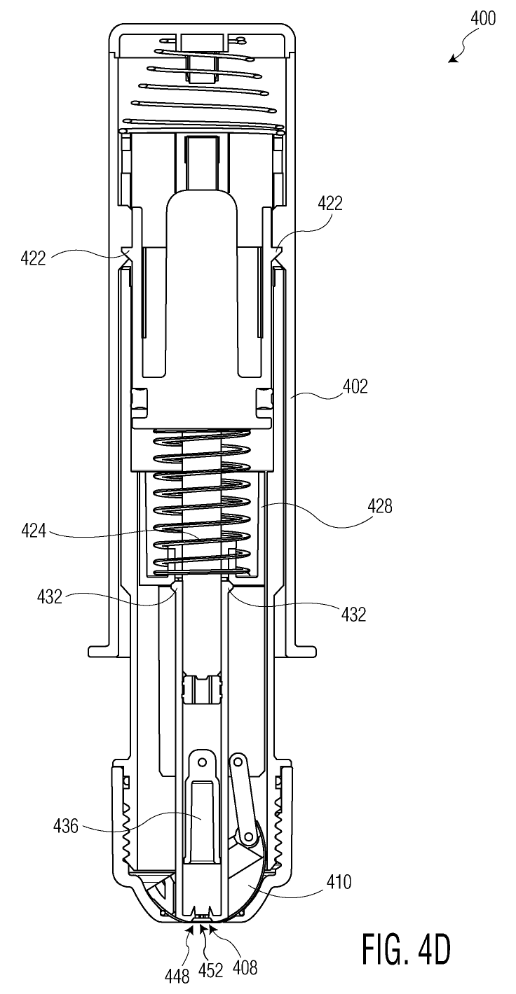

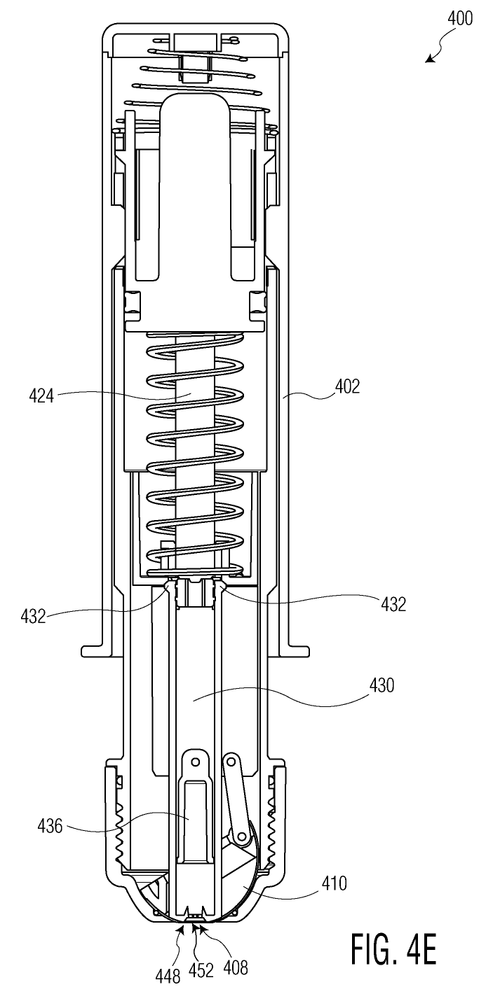

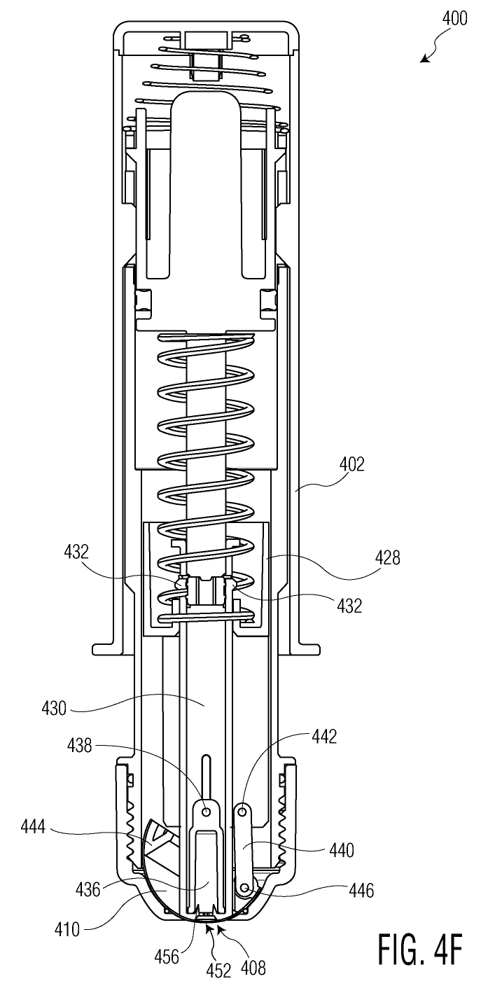

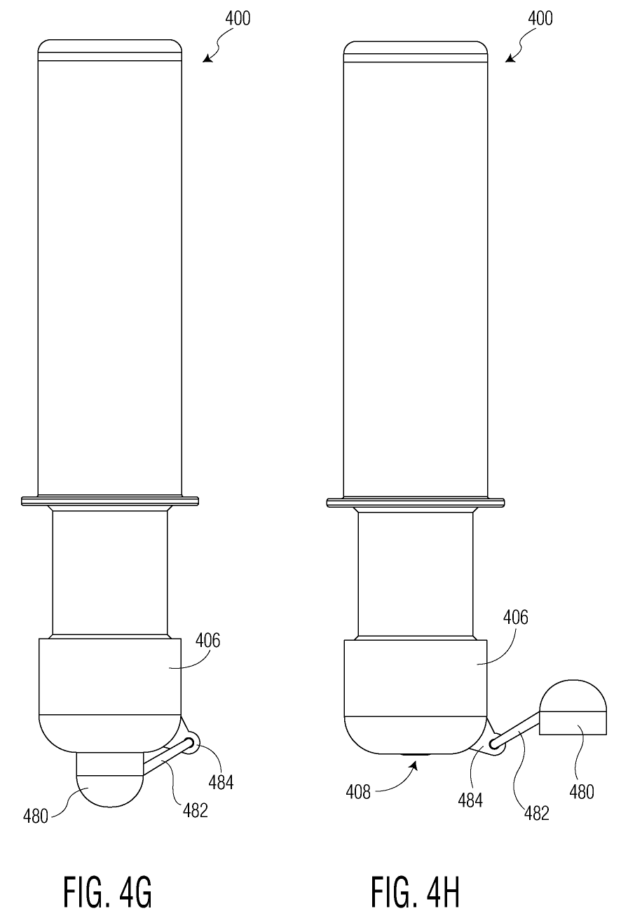

Referring now to FIGS. 4A-4H, diagrams of a shave biopsy device 400 for collecting a shave biopsy sample according to another example are shown.

FIG. 4A is a perspective view of the device 400 in an unactuated state. FIG. 4B is a perspective view of the device 400 in an actuated state. The device 400 may include a handle 402, a top 403, body 404, and a tip 406 with an opening 408, and a blade 410. The tip 406 may be located at a first end of the device 400 and the top 403 may be located at a second end of the device 400.

The handle 402 may form upper cylindrical portion configured to be gripped by a user and may serve as an actuator. The handle 402 may have a first end 402A (e.g. an “upper” or “up” end) and a second end 402B (e.g., a “lower” or “down” end that is closer to a patient). The body 404 may be disposed below the handle 402 (i.e., closer to a patient) and may house internal actuation components discussed below. The tip 406 may be disposed at a distal end of the body 404 (i.e., closer to a patient) and may be in a rounded or dome-shaped configuration. The opening 408 may be disposed at a distal end of the tip 406 and may be configured to receive a tissue from the patient for biopsy collection. In the unactuated state shown in FIG. 4A, the opening 408 may be exposed and ready to be positioned over a region of interest on a tissue. In the actuated state, shown in FIG. 4B, the blade 410 may have been actuated to cut a tissue sample and seal the opening 408. The blade 410 may enclose the collected tissue sample within the device 400, thereby maintaining sample integrity and preventing contamination after the tissue has been excised.

FIG. 4C is a cross-section view of the device 400 in the unactuated state (i.e., “position 1”). As shown, a first portion of the body 404 may fit within a portion of the handle 402 via the second end 402B. An interior of the handle 402 may have handle protrusions 412 that extend towards a center of the handle. The body 404 may be shaped such that the first protrusions allow the body 404 to move towards the first end 402A but not toward the second end 402B. A second portion of the body 404 may fit within a portion of the tip 406. The second portion of the body 404 may be secured within the tip 406 by a first O-ring 414. The first O-ring 414 may fit within cavities of the body 404 and may secure the body 404 in place with relation to the tip 406.

The top 403 may be secured to the handle 402 via a catch 405 to prevent movement. The catch 405 may be a protrusion that extends from the tip 406 into a grove formed into an interior of the handle 402. A first spring 416 may be secured by the top 403 and the handle protrusions 412. The first spring 416 may be a return spring to keep the handle 402 from being accidentally compressed. In the unactuated state (i.e., “position 1”), the first spring 416 may be partially compressed. A second spring 418 may be secured by a piston 420 and a carriage 428. In the unactuated state (i.e., “position 1”), the second spring may be fully compressed.

The piston 420 may be held in place within the body 404 by piston snaps 422. The piston snaps 422 may be protrusions that extend from the piston into groves formed into an interior of the body 404. The piston 420 may be coupled to a plunger 424. In an example, the piston 420 and the plunger 424 may be an integrated component. The piston 420 may include grooves that hold a second O-ring 426 in place between the piston 420 and the body 404.

The plunger 424 may pass through an opening of the carriage 428 that houses the second spring 418 and may extend into a sample tube 430. The carriage 428 may be secured in place by carriage snaps 432 that extend from an outer surface of the sample tube 430. The sample tube 430 may extend through the body 404 and into the tip 406. An end of the sample tube 430 may include the opening 408. A blocking plate 451 may block a degree of ingress of tissue into the opening 408. An interior perimeter of a tissue chamber 452 may include one or more protrusions 456. The one or more protrusions 456 may be configured to pierce a covering 434 of a fluid container 436 located within the sample tube 430. The fluid container 436 may contain one or more of a fixative, a preservative, a cell culture medium, a growth medium, formalin, and additional types of mediums that may be useful for storing a biopsy sample. In an example, the covering 434 may be a foil or other impermeable membrane. The fluid container 436 may be coupled to the carriage 428 via a first dowel pin 438 that extends through an opening in the sample tube 430.

The carriage 428 may also be coupled to a first end of linkage 440 via a second dowel pin 442. A second end of the linkage 440 may be coupled to a cutter body 444 via a third dowel pin 446. The cutter body 444 may be coupled to the blade 410. In an example, the blade 410 may be hemispherical in shape. The blade 410 may have a hole 448. In an example, a diameter of the hole 448 may be equal to or greater than a diameter of the opening 408. The blade 410 may sit on a third O-ring 450 fitted into a groove formed in the distal end of the tip 406. A tissue chamber 452 may be defined by the opening 408.

In the unactuated state (i.e., “position 1”) shown in FIG. 4C, the handle 402 may in be in an up position. The piston 420 and plunger 424 may be in a down position compressing the second spring 418. The carriage 428 may be in an up position held in place by the carriage snaps 432. The fluid container 436 may be in an up position within the sample tube 430. The blade 410 may be retracted, such that the hole 448 is aligned with the opening 408.

FIG. 4D is a cross-section view of the device 400 in a first partially actuated state (i.e., “position 2”) in which the handle 402 has been partially pressed down an initial amount. The device 400 may be pressed against a tissue of a patient. This may cause the piston snaps 422 to release. The plunger 424 may be ready to spring upwards. The carriage 428 may still be in the up position held in place by the carriage snaps 432. The fluid container 436 may be in the up position within the sample tube 430. The blade 410 may be retracted, such that the hole 448 is aligned with the opening 408. The first spring 416 may be fully compressed and the second spring 418 may be fully compressed.

FIG. 4E is a cross-section view of the device 400 in a second partially actuated state (i.e., “position 3”) in which the handle 402 has been partially pressed down an additional amount. The device 400 may still be pressed against the tissue of the patient. This may cause the plunger 424 to spring upwards (e.g., driven by the second spring 418), creating a vacuum within the sample tube 430. The fluid container 436 may have a diameter that is smaller than a diameter of the sample tube 430, such that the vacuum causes suction at the opening 408. The tissue of the patient may be secured in place within the tissue chamber 452 and/or drawn into the tissue chamber 452 as a result of the vacuum. The carriage snaps 432 may be at the point of release and the carriage 428 may be ready to spring downwards. The fluid container 436 may be in the up position within the sample tube 430. The blade 410 may be retracted, such that the hole 448 is aligned with the opening 408. The first spring 416 may be fully compressed and the second spring 418 may be partially uncompressed.

FIG. 4F is a cross-section view of the device 400 in the actuated state (i.e., “position 4”) in which the handle 402 has been fully pressed down. The first spring 416 may be fully compressed and the second spring 418 may be almost fully uncompressed. This may cause the carriage 428 to spring downwards. The downward movement of the carriage 428 may be transferred to the fluid container 436 through the first dowel pin 438, causing the fluid container 436 to move down through the sample tube 430. At the same time, the downward movement of the carriage 428 may be transferred to the linkage 440 via the second dowel pin 442, causing the linkage 440 to move down. The movement of the linkage 440 may then be transferred to the cutter body 444 via the third dowel pin 446. The movement of the cutter body 444 may cause the blade 410 to move, such that the hole 448 moves laterally across the opening 408. This may cause the blade 410 to cut the tissue that has been drawn into the opening 408 and then seal the tissue chamber 452 shut with a tissue sample inside. As the fluid container 436 moves downwards, the covering 434 may come into contact with the one or more protrusions 456, and the covering 434 may be pierced. The fluid container 436 may come in contact with the sample tube 430, thereby sealing the tissue sample and the one or more of the fixative, the preservative, the cell culture medium, the growth medium, formalin, and additional types of mediums within the opening 408 sealed by the blade 410.

FIGS. 4G-4H show an alternative embodiment of the device 400 that includes a lid 480 configured to cover the opening 408. FIG. 4G is a planar view of the device 400 showing the lid 480 in a closed position. FIG. 4H is a planar view of the device 400 showing the lid 480 in an open position. The lid 480 may be coupled to the tip 406 via a hinge 482. In an example, the lid 480 may contain the storage medium. Closure of the lid 480 over the end of the device 400 may cause the storage medium to become in continuity with the tissue chamber 452 and simultaneously seal the device 400. A first end of the hinge 482 may be connected to a mount 484 on the tip 406. A second end of the hinge 482 may be connected to the lid 480.

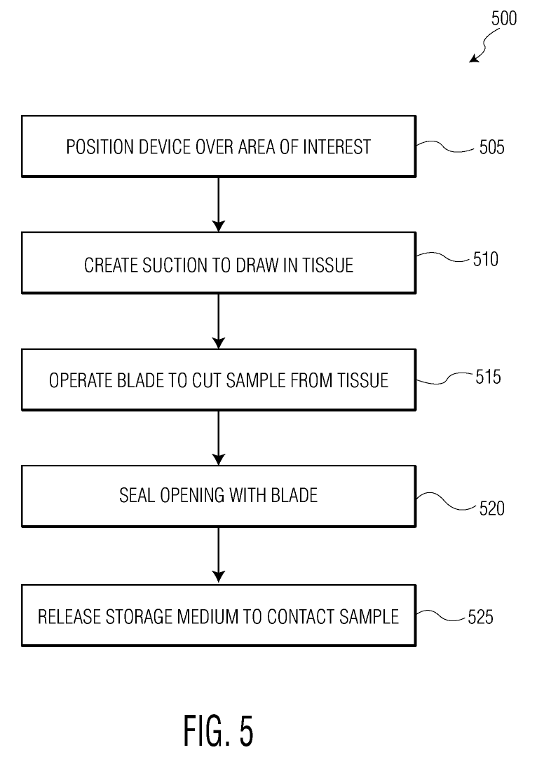

Referring now to FIG. 5, a flow chart of an illustrative method 500 of collecting a shave biopsy sample using a biopsy device, including any one of device 100, device 200, device 300, and device 400 is shown. At step 505, the device may be positioned over an area of interest. At step 510, suction may be created to draw issue of the area of interest into an opening of the device. At step 515, a blade may be operated to cut a sample from the tissue. At step 520, the opening may be sealed with the blade. A step 525, a storage medium may be released into the sealed space to contact the sample therewith.

The device may be placed over an area of interest (e.g., a lesion) by aligning the opening with the tissue such that the area of interest is within the opening. The tissue may be prepared for biopsy in a variety of manners. For example, a dermal tissue may be prepared by cleaning the area, e.g., with alcohol. Furthermore, the dermal tissue may be prepared by anesthetizing, e.g., with a transdermal lidocaine patch, an injection, or another anesthetic as would be known to a person having an ordinary level of skill in the art.

Suction may be created by withdrawing a plunger to draw to the tissue into the opening. In some examples, an actuator may be triggered to cause the plunger to withdraw, thereby generating the suction at the opening. A blocking plate may be perforated to allow the suction force to pass therethrough to act on the tissue. The tissue may be drawn in up to the blocking plate and compressed against the blocking plate to a predetermined depth based on the position of the blocking plate.

The blade may be operated to move across the opening, thereby cutting a sample from the tissue while the tissue is drawn into the tissue chamber by the suction from the plunger. The blade may be spring loaded. The blade may be configured to automatically release based on the suction provided by the plunger. For example, when the plunger is withdrawn a certain distance, the blade mechanism may release the blade and the force of the spring-loaded mechanism may cause the blade to move from a first position to a second position, thereby cutting the sample from the tissue. In some examples, the blade may transect the tissue in a flat plane (e.g., parallel to the blocking plate) to obtain the sample at the predetermined depth. The blade may be received in a slot in the second position to retain the blade in the second position and/or seal the opening as described herein.

The blocking plate may be withdrawn to open additional portions of the tissue chamber to receive the sample. The blocking plate may have a cylindrical column that is retained by one or more retaining pins that extend towards the sides of the housing. The retaining pins may be released by drawing the retaining pins away from the sides of the housing. Accordingly, the blocking plate may be released from its fixed position. The blocking plate may then be withdrawn and the sample may advance further into the tissue chamber.

The storage medium may be released from a fluid chamber to contact the sample. In some examples, the release of the storage medium may occur automatically. For example, the movement of the blocking plate may trigger a mechanism to release the storage medium. The fluid chamber may be pressurized or spring-loaded such that the movement of the blocking plate causes the fluid chamber to release the storage medium. Accordingly, the sample may be safely stored for transport to a testing facility or another facility for examination and/or assessment.

The devices, systems, and methods as described herein are not intended to be limited in terms of the particular examples described, which are intended only as illustrations of various features. Many modifications and variations to the devices, systems, and methods can be made without departing from their spirit and scope, as will be apparent to those skilled in the art.

While exemplary storage mediums are described herein, it is envisioned that any variety of storage media suitable for storing a tissue sample may be utilized with the devices and methods herein as would be known to a person having an ordinary level of skill in the art.

Exemplary mechanisms are described herein (e.g., blade mechanisms, mechanisms for the blocking plate, mechanisms for releasing the storage medium, cam mechanisms, actuator mechanisms, actuator types, and the like. It is envisioned that the mechanisms could be designed in a variety of manners to accomplish the results described herein as would be known to a person having an ordinary level of skill in the art. Accordingly, such exemplary mechanisms are not intended to be limiting.

The devices described herein are shown, for example, in the general shape and design of a syringe. However, it should be understood that these designs are exemplary and are not intended to be limiting. The form factor of the devices described herein could be modified in a variety of manners as would be known to a person having an ordinary level of skill in the art.

In the above detailed description, reference is made to the accompanying drawings, which form a part hereof. In the drawings, similar symbols typically identify similar components, unless context dictates otherwise. The illustrative examples described in the present disclosure are not meant to be limiting. Features and aspects of the examples described herein may be combined with one another, other examples may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular examples described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular examples described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular examples only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed examples.

What is claimed is:

1. A device for collecting a biopsy sample, the device comprising:
a first end comprising a tissue chamber in fluid communication with an opening configured to receive a tissue, such that the tissue extends at least partially into the tissue chamber;
a fluid chamber containing a storage medium, the fluid chamber in fluid communication with the tissue chamber;
a blade adjacent the opening; and
an actuator that, when triggered by a user input, is configured to cause:
the blade to move from a first position in which the opening is unobstructed, across the opening, and to a second position in which the opening is obstructed by a portion of the blade, thereby cutting a sample from the tissue and sealing the sample within the tissue chamber, and
the fluid chamber to release the storage medium into the tissue chamber when the blade is in the second position.

2. The device of claim 1, further comprising:
a plunger in fluid communication with the tissue chamber; and
a blocking plate within the tissue chamber, the blocking plate configured to contact the tissue.

3. The device of claim 2, wherein the actuator, when triggered by the user input, is further configured to cause the plunger to move thereby providing suction at the opening to draw the tissue through the opening and into the tissue chamber.

4. The device of claim 3, wherein the blocking plate is perforated, thereby enabling the plunger to provide the suction at the opening.

5. The device of claim 3, wherein movement of the plunger causes the fluid chamber to release the storage medium into the tissue chamber.

6. The device of claim 2, wherein the actuator, when triggered by the user input, is further configured to cause the blocking plate to move with respect to the opening.

7. The device of claim 5, wherein movement of the blocking plate causes the fluid chamber to release the storage medium into the tissue chamber.

8. The device of claim 1, wherein the first end comprises a slot configured to receive the blade in the second position.

9. The device of claim 1, wherein the fluid chamber comprises a covering.

10. The device of claim 9, wherein the actuator when triggered by the user input, is further configured to cause the fluid chamber to move such that the covering is pierced by one or more protrusions on an interior perimeter of the tissue chamber, thereby causing the release of the storage medium into the tissue chamber.

11. The device of claim 1, wherein the blade comprises a hole that is aligned with the opening in the first position.

12. A method of biopsying a tissue, the method comprising:
positioning a biopsy device over a region of interest comprising the tissue, the biopsy device comprising:
an opening at a first end configured to receive the tissue such that the tissue extends at least partially into a tissue chamber,
a fluid chamber containing a storage medium, the fluid chamber in fluid communication with the tissue chamber,
a blade adjacent the opening, and
an actuator; and
triggering the actuator via a user input causing:
the blade to move from a first position in which the opening is unobstructed, across the opening, and to a second position in which the opening is obstructed by a portion of the blade, thereby cutting a sample from the tissue and sealing the sample within the tissue chamber, and
the fluid chamber to release the storage medium into the tissue chamber when the blade is in the second position.

13. The method of claim 12, wherein the triggering the actuator via the user input further causes one or more of a blocking plate to move with respect to the opening and a plunger in fluid communication with the tissue chamber to move.

14. The method of claim 13, wherein movement of the plunger provides suction at the opening to draw the tissue through the opening and into the tissue chamber.

15. The method of claim 14, wherein the blocking plate is perforated, thereby enabling the plunger to provide the suction at the opening.

16. The method of claim 13, wherein the blocking plate is configured to contact the tissue.

17. The method of claim 13, wherein movement of the blocking plate causes the fluid chamber to release the storage medium into the tissue chamber.

18. The method of claim 12, wherein the triggering the actuator via the user input further causes the blade to be received within a slot of the first end in the second position.

19. The method of claim 12, wherein the triggering the actuator via the user input further causes the fluid chamber to move such that a covering of the fluid chamber is pierced by one or more protrusions on an interior perimeter of the tissue chamber, thereby causing the release of the storage medium into the tissue chamber.

20. The method of claim 12, wherein the triggering the actuator via the user input further causes a hole of the blade that is aligned with the opening in the first position to move across the opening.
